(19) United States
(12) Patent Application Publication
(10) Pub. No.: US 2025/0318821 A1
(43) Pub. Date: Oct. 16, 2025

(54) TISSUE ENGAGING DEVICES, SYSTEMS, AND METHODS

(71) Applicant: Boston Scientific Scimed, Inc., Maple Grove, MN (US)

(72) Inventor: Kevin L. Bagley, Natick, MA (US)

(73) Assignee: Boston Scientific Scimed, Inc., Maple Grove, MN (US)

(21) Appl. No.: 19/178,160

(22) Filed: Apr. 14, 2025

Related U.S. Application Data
(60) Provisional application No. 63/634,181, filed on Apr. 15, 2024.

(51) Int. Cl. A61B 17/02; A61B 17/00; A61B 17/32; A61M 13/00

(52) U.S. Cl. CPC A61B 17/0218; A61B 17/320016; A61M 13/003; A61B 2017/00269; A61B 2017/00557; A61B 2017/320044

(57) ABSTRACT

A device, system, and method for engaging tissue and/or applying traction to tissue. A first component of a tissue-engaging device engages a first section of tissue while a second component of the tissue-engaging device engages a second section of tissue spaced apart from the first section of tissue. The first component may be configured to grasp target tissue at the first section, whereas the second component may be configured to simply engage, but not grasp, anchor tissue at the second section. Thus, the second component may grasp anchor tissue with the assistance of the second component, but may be readily removed from the anchor tissue without damaging the anchor tissue while the target tissue remains grasped by the second component.

### CROSS-REFERENCE TO RELATED APPLICATIONS

This application claims the benefit of priority under 35 U.S.C. § 119 to U.S. Provisional Application No. 63/634,181, filed Apr. 15, 2024, the entire disclosure of which is hereby incorporated by reference herein for all purposes.

### FIELD

The present disclosure relates generally to the field of medical devices, systems, and methods for engaging tissue. In particular, the present disclosure relates to medical devices having different components for engaging tissue in more than one manner, and associated systems and methods. Even more particularly, the present disclosure relates to medical devices, and associated systems and methods, in which a first component of the medical device is shaped to interact with tissue in first manner, and a second component of the medical device is shaped to interact with tissue in a second, different manner, independently of the first component. The devices, systems, and methods are particularly suitable for use in a body lumen.

### BACKGROUND

A number of medical procedures, including but not limited to those performed along the digestive and/or biliary tract, utilize medical devices to access tissue intended for removal (e.g., “target tissue”) from within the body. Various such procedures require maneuvering about various anatomical structures. Some procedures, such as endoscopic mucosal resection (EMR), Endoscopic Submucosal Dissection (ESD), Per-Oral Endoscopic Myotomy (POEM), cholecystectomy, Video-Assisted Thoracoscopic Surgery (VATS)), etc., allow for minimally invasive removal of tissue (e.g., benign as well as lesions such as early malignant lesions), such as in the gastrointestinal (GI) tract, by utilizing a medical scope or similar medical device during access and removal of the tissue. The medical scope or other delivery device may be capable of both accessing the target tissue site (the site at which the target tissue is located) while also permitting various tissue manipulating devices to be deployed therethrough. Because such procedures are minimally invasive, there is limited space to maneuver within the body. In procedures involving cutting of tissue, the loose section of tissue (cut with respect to surrounding tissue) may obstruct visibility, such as by falling on the visualization device being used during the procedure and occluding visibility, and/or may otherwise create a hindrance affecting movement of the instruments used during the procedure (e.g., obstructing the ability to reach the extreme corners of the target tissue being cut). These complications may directly contribute to increased procedures time, complexity and risk of perforation or bleeding. Various solutions for lifting the cut (e.g., hanging mass) of tissue, thus clearing the path for visibility and operation of medical tools and devices, have been developed. However, positioning and maneuvering the elements used with such solutions may be challenging. Also, the elements used with such solutions may require separate medical tools than those used to perform the procedure, and such tools may even require a separate working channel in the delivery device (e.g., endoscope), thereby potentially increasing the size and/or complexity of the delivery device. There remains a need for alternative solutions for lifting tissue and/or applying traction to tissue during a procedure, and solutions to associated challenges, which reduce cost, complexity, length of procedure time, and cognitive load presented by currently available solutions would be welcome.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a method of applying traction to tissue within a patient's body lumen includes grasping, with the same tissue-engaging device, target tissue at a target section along a wall of the body lumen and anchor tissue at an anchor section along a wall of the body lumen spaced apart from the target section; and insufflating the body lumen to move the anchor section apart from the target section while the tissue-engaging device remains grasping both sections to apply traction to the target section.

In some aspects, the method further includes cutting tissue adjacent the target tissue to separate the target tissue from surrounding tissue, and further insufflating the body lumen to separate the anchor section further from the target section to increase tension on the target tissue.

In some aspects, the method further includes insufflating the body lumen to advance the tissue-engaging device to the target tissue, and deflating the body lumen sufficiently to allow a first component of the tissue-engaging device to grasp the target tissue and a second component of the tissue-engaging device to engage the anchor tissue. In some aspects, the method further includes deflating the body lumen sufficiently to allow the second component to form a fold in the anchor section to define the anchor tissue. In some aspects, the method further includes cutting tissue at the target section to separate the target tissue from surrounding tissue, wherein the first component of the tissue-engaging device is configured to grasp and remain engaged with the target tissue, and the second component of the tissue-engaging device is configured to be slid off the anchor tissue.

In accordance with various principles of the present disclosure, a method of engaging two different areas of tissue in a patient's body in different manners is performed using the same tissue-engaging device. The method includes grasping a first tissue section with a first component of the tissue-engaging device to grasp the first tissue section in a first direction and to remain engaged therewith when moved transverse to the first direction; engaging a second tissue section, spaced apart from the first tissue section, with a second component of the tissue-engaging device to engage the second tissue section without restricting movement of the second component with respect to the second tissue section; and causing movement of the second tissue section to be restricted with respect to the second component with the use of a component of the tissue-engaging device other than the second component.

In some aspects, the method further includes grasping the second tissue section by moving the first component closer to the second component to grasp the second tissue section therebetween. In some aspects, the method further includes cutting the first tissue section away from surrounding tissue; and removing the tissue-engaging device from the patient's body by sliding the tissue-engaging device with respect to the second tissue section to remove the second component from engagement therewith without damaging the second tissue section, and maintaining the first tissue section grasped by the first component.

In accordance with various principles of the present disclosure, a tissue-engaging device includes a first component shaped, contoured, and/or configured to grasp a first tissue section to remain in place with respect to the first component; and a second component shaped, contoured, and/or configured to engage a second tissue section while remaining movable tangentially with respect to the engaged second tissue section. In some aspects, the first component and the second component are movable with respect to each other to move the first component into proximity with the second component to grasp the second tissue section between the second component and the first component to limit tangential movement of the second tissue section with respect to the second component.

In some aspects, the first component is shaped to engage tissue in a first direction and to be restricted from moving in a second direction transverse to the first direction. In some aspects, the first direction is transverse to the contact surface of the tissue. In some aspects, the first component is shaped to penetrate the tissue along the first direction.

In some aspects, the first component includes a tissue-grasping feature in the form of a tissue-penetrating feature, and the second component has a blunt tissue-engagement feature.

In some aspects, the first component is curved.

In some aspects, the second component is shaped to remain movable with respect to the second tissue section in both the first direction and the second direction.

In some aspects, the first component is defined along a first tissue-engagement member, and the second component is defined along a second tissue-engagement member, the first tissue-engagement member and the second tissue-engagement member being movable with respect to each other between a first configuration in which tissue may be received between the first tissue-engagement member and the second tissue-engagement and a second configuration in which the first tissue-engagement member holds tissue with respect to the second tissue-engagement member to grasp tissue between the first tissue-engagement member and the second tissue-engagement member.

In some aspects, the first tissue-engagement member and the second tissue-engagement member are pivotably coupled with respect to each other.

In some aspects, the first component is defined along a distal end of the first tissue-engagement member, and the second component is defined along a distal end of the second tissue-engagement member.

In some aspects, the first component is shaped to penetrate tissue in a direction transverse to the contact surface of the first tissue section. In some aspects, the first component is shaped to retain the first tissue section grasped therewith upon cutting the first tissue section away from surrounding tissue.

In some aspects, the tissue-engaging device disclosed herein is included in a tissue-engaging system which also includes a delivery device. In some aspects, the tissue-engaging device is separable from the delivery device. In some aspects, the system further includes a cutting device for cutting around the first tissue section.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope or the like. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates an example of an embodiment of a tissue-engaging device formed in accordance with aspects of the present disclosure.

FIG. 1A illustrates a detail view of an alternative configuration of a component of the tissue-engaging device illustrated in FIG. 1.

FIG. 2 illustrates a schematic view of a tissue-engaging device formed in accordance with various principles of the present disclosure being advanced to target tissue within a body lumen of a patient.

FIG. 3A illustrates a schematic view of a component of the tissue-engaging device of FIG. 2 initially grasping target tissue within a body lumen.

FIG. 3B illustrates a schematic view similar to that of FIG. 3A, but with the body lumen deflated to allow the tissue-engaging device to engage an anchor section of the body lumen wall spaced apart from the target tissue section of the body lumen wall.

FIG. 3C illustrates a schematic view of a component of the tissue-engaging device illustrated in FIG. 3B forming a fold in the anchor section of the body lumen wall to define anchor tissue.

FIG. 3D illustrates a schematic view similar to that of FIG. 3C, but with the body lumen further deflated to allow the tissue-engagement members of the illustrated example of an embodiment of a tissue-engaging device to be moved to a closed, tissue-grasping configuration.

FIG. 3E illustrates a schematic view of cutting of the target tissue, and insufflation of the body lumen illustrated in FIG. 3D to increase traction on the target tissue.

FIG. 3F illustrates a schematic view similar to that of FIG. 3E, but with the target tissue further cut and the body lumen further insufflated to increase traction on the target tissue.

FIG. 3G illustrates a schematic view similar to that of FIG. 3F but with the cutting device reaching the full extent of tissue to be cut from the target tissue section of the body lumen wall.

FIG. 3H illustrates a schematic view similar to that of FIG. 3F, but with the target tissue fully cut away from the target tissue section of the body lumen, and remaining grasped by the tissue-engaging device which remains grasping the anchor tissue as well.

FIG. 3I illustrates a schematic view of a body lumen such as illustrated in FIG. 3F, but with the tissue-engaging device and the target tissue proximally withdrawn, allowing the anchor tissue to return to its prior configuration.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

The present disclosure relates to devices, systems, and methods for engaging and/or applying traction to tissue within a body. Various traction devices, systems, and methods utilize tissue traction devices or systems coupled with tissue at a target tissue site with the use of a tissue-engagement member. The tissue-engagement device, which may alternately be referenced herein as a tissue fastener or clip or other mechanical securing device (e.g., a hemostatic clip, clamp, grasper, basket, gripper, magnet, adhesive, etc.), without intent to limit, is configured to engage the target tissue as traction is applied to the target tissue. It will be appreciated that terms such as engage (and other grammatical forms thereof) may be used interchangeably herein with terms such as contact, touch, abut, etc., without intent to limit, to indicate a physical interaction between an element and another element. In some aspects, a component of the tissue-engaging device is configured to grasp the tissue to maintain the tissue therewith. It will be appreciated that terms such as grasp (and other grammatical forms thereof) may be used interchangeably herein with terms such as couple, hold, clasp, clip, anchor, etc. (and other grammatical forms thereof), without intent to limit, to indicate a physical interaction between an element and another element which maintains the elements in close proximity (e.g., in contact) such that at least one of the elements moves when the other of the elements is moved. It will be appreciated that the term engage does not necessarily encompass grasping, but the term grasp encompasses engaging.

Prior traction devices or systems have included an elastic and/or stretchable element which is coupled to tissue such as with the use of one or more tissue-engagement devices (e.g., with a tissue-engagement device at each end of the elastic element). In accordance with various principles of the present disclosure, a tissue-engagement device is used without an additional element to apply traction to target tissue within a body passage as the target tissue is cut (dissected, resected, etc.). Instead, initial engagement of the tissue-engagement device with the target tissue and with an anchor tissue is achievable when the body passage is substantially collapsed (typical during a gastrointestinal procedure when the intestines have been cleared by a pre-procedure preparation) and the opposed tissue walls are relaxed (e.g., not taut) and in sufficiently close proximity for the tissue-engagement member to engage the opposed tissues. Insufflation of the body passage facilitates movement of the opposed tissue walls apart from one another with the tissue-engagement device coupled to both tissue walls, thereby apply traction to the target tissue by engagement of the tissue-engagement device with the anchor tissue.

In accordance with various principles of the present disclosure, insufflation may be used to provide dynamic aspects to traction that are currently underutilized. More particularly, devices, systems, and methods of the present disclosure are particularly suitable for use in a body lumen (e.g., a tubular body passage, such as within the intestines). However, it will be appreciated that principles of the present disclosure may be applicable to other anatomical structures, such as body cavities, organs (e.g., the stomach), passages, etc. For the sake of convenience, and without intent to limit, reference is made to body lumens, with the understanding that descriptions with respect to body lumens are applicable to other anatomical structures or passages. After the body lumen has been cleared of materials in preparation for the procedure, opposed sides of the lumen (e.g., diametrically opposed locations along the lumen wall) may be close enough, or at least the lumen wall may be sufficiently flaccid or at least manipulable, for a tissue-engagement device to span across the lumen from one side of the lumen wall to another (e.g., opposite) side. A tissue-engagement device formed in accordance with various principles of the present disclosure may be advanced to the target site. In some embodiments, the tissue-engagement device has two or more tissue-engagement members (e.g., grasper arms or jaws) movable between a closed configuration (e.g., for delivery to a target tissue site and/or to grasp tissue) and an open configuration (to receive tissue to be grasped by the tissue-engaging device between the tissue-engagement members). In some aspects, the grasper arms are coupled together and movable with respect to each other. The grasper arms may be formed as a single piece, or may be separately formed, and movable such as by being hinged together, pivotably movable about a pivot point (a common pivot point, or a respective pivot point for each grasper arm), etc. In some aspects, the tissue-engaging device is movable between the open and closed configurations multiple times to allow repositioning. In some aspects, the tissue-engagement device may be configured to allow for the tissue-engagement device to be locked against movement opening out of engagement with tissue when in a closed configuration.

In accordance with various principles of the present disclosure, a first component of the tissue-engagement device may be engaged with tissue at a first location within the body lumen, and a second component of the tissue-engagement device may be engaged with tissue at a second location within the body lumen spaced apart from the first location. It will be appreciated that although such engagement may be indirect, devices, systems, and methods of the present disclosure are simplified by such engagement being a direct engagement. In some aspects, one of the first location or the second location is the target tissue, and the other of the first location or the second location is anchor tissue spaced apart from the target tissue. Engagement of a tissue-engagement device with the first location and the second location allows for traction to be applied to the target tissue as the lumen is insufflated. In particular, in accordance with various principles of the present disclosure, the body lumen is insufflated to separate (e.g., increases the distance between) the first location and the second location. With the tissue-engagement device coupled to both the first location and the second location, as the target tissue is cut and separated from surrounding tissue, and as the body lumen is insufflated to separate the anchor tissue from the tissue immediately surrounding the target tissue, the target tissue is moved with the anchor tissue to be lifted away from the tissue immediately surrounding the target tissue.

In accordance with various principles of the present disclosure, a tissue-engaging device and associated system are designed to facilitate performance of the novel technique for applying traction to tissue described herein. In some aspects, the tissue-engaging device has a first tissue-engagement member and a second tissue-engagement member which do not have the same shape, contour, configuration, dimensions, etc. Instead, the first tissue-engagement member and the second tissue-engagement member are shaped, contoured, configured, dimensioned, etc., differently to engage tissue in different manners. In some aspects, the differently shaped first and second tissue-engagement members are designed and adapted for different physical interaction with tissue. For instance, the first tissue-engagement member may be shaped, contoured, and/or configured to securely engage tissue, such as to grasp the tissue such as by penetrating into the tissue. Thus, as traction is applied to the target tissue, the tissue-engagement member grasping the target tissue remains engaged therewith. The other of the tissue-engagement members may be shaped, contoured, and/or configured to releasably engage anchor tissue to facilitate removal therefrom, such as once the target tissue has been completely separated from immediately-surrounding tissue, without damaging the anchor tissue. In some aspects, the tissue-engagement members are movable between an “open” configuration apart from each other, and a “closed” configuration as close as possible to each other with the target tissue and anchor tissue grasped between the tissue-engagement members. In some aspects, the tissue-engagement members are in the form of jaws which move towards and away from each other. The tissue-engagement members may be moved together to grasp the target tissue along with the anchor tissue between the first and second tissue-engagement members. In the closed configuration the configuration of the tissue-engagement member engaged with the anchor tissue may facilitate engagement without slippage as the body lumen is insufflated. To facilitate release of the tissue-engaging device once the target tissue has been completely separated from immediately-surrounding tissue, the tissue-engagement member engaged with the anchor tissue may be shaped and configured to be secured with respect to the anchor tissue when adjacent the other tissue-engagement member, yet to be slidable with respect to the anchor tissue when pulled proximally therefrom.

Various embodiments of devices, systems, and methods for engaging tissue and/or applying traction to tissue will now be described with reference to examples of embodiments illustrated in the accompanying drawings. It will be appreciated that the following description is of illustrative examples of embodiments only, and is not intended as limiting the broader aspects of the present disclosure.

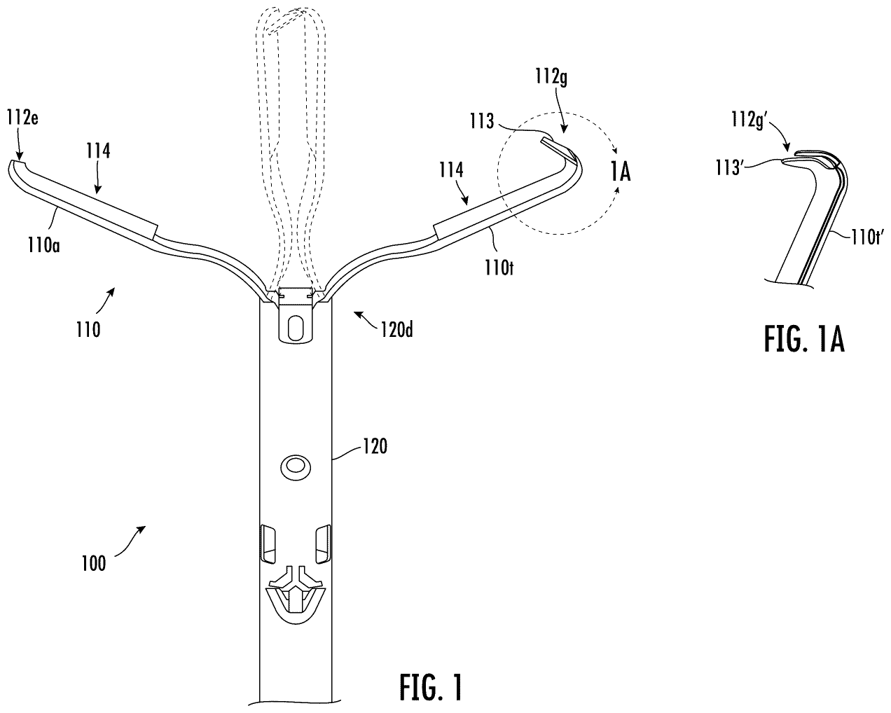

Turning now to the drawings, an example of an embodiment of a tissue-engaging device 100 formed in accordance with various principles of the present disclosure is illustrated in FIG. 1. The illustrated example of an embodiment of a tissue-engaging device 100 has a first tissue-engagement member 110t and a second tissue-engagement member 110a. In some aspects, the first tissue-engagement member 110t and the second tissue-engagement member 110a are movable with respect to each other, such as pivotably coupled with respect to each other and with respect to a shaft 120. In some aspects, the first tissue-engagement member 110t and a second tissue-engagement member 110a are movable between an open configuration (as illustrated in solid lines in FIG. 1), and a closed configuration (as illustrated in broken lines in FIG. 1). In the open configuration, the tissue-engagement member 110t and the second tissue-engagement member 110a may be spaced apart sufficiently to receive tissue therebetween. In the closed configuration, the tissue-engagement member 110t and the second tissue-engagement member 110a may be close enough to each other to grasp tissue therebetween. In some aspects, the tissue-engaging device 100 may be considered a tissue-grasping device.

In accordance with various principles of the present disclosure, the tissue-engaging device is shaped, contoured, configured, and/or dimensioned to be able to engage with tissue in more than one manner. More particularly, the tissue-engaging device 100 includes two or more components, with at least one of the components having a shape, contour, configuration, and/or dimensions different from those of another component. Such components are differently shaped, contoured, configured and/or dimensioned so that they may interact with tissue in different manners. The tissue-engaging device 100 is thus capable of interacting with tissue in more than one manner. For instance, a first component is capable of being moved with respect to tissue in a first manner, and a second component is capable of being moved with respect to tissue in a second manner. In some aspects, the first component and the second component are movably coupled with respect to each other.

In some aspects, the differently shaped, contoured, configured, and/or dimensioned components are associated with the tissue-engagement member 110 of the tissue-engaging device 100, such as with a first tissue-engagement member 110t and a second tissue-engagement member 110a of the tissue-engaging device 100. For instance, the first tissue-engagement member 110t and/or a limited component or portion thereof (such as the distal end thereof, as described in further detail thereof) may be considered a first component which is shaped, contoured, configured, and/or dimensioned differently from another component of the tissue-engaging device 100. Alternatively or additionally, the second tissue-engagement member 110a and/or a limited component or portion thereof (such as the distal end thereof, as described in further detail thereof) may be considered a second component which is shaped, contoured, configured, and/or dimensioned differently from the first component of the tissue-engaging device 100. In some aspects, the first tissue-engagement member 110t and the second tissue-engagement member 110a of the tissue-engaging device 100 are movably coupled with respect to each other, with the respective first and second components thereby being movably coupled with respect to each other.

As may be appreciated, with reference to the example of an embodiment illustrated in FIG. 1, the first tissue-engagement member 110t and the second tissue-engagement member 110a are not identical and are not even symmetrical/mirror images of each other.

Instead, the first tissue-engagement member 110t and the second tissue-engagement member 110a have different shapes, contours, configurations, dimensions, etc. The different shapes, contours, configurations, dimensions, etc., of the tissue-engagement members 110t, 110a are structured to engage with tissue in different manners to allow the tissue-engagement members 110t, 110a to engage and/or interact with tissue in different manners, such as to perform procedures requiring different manipulations of tissue, as described in further detail below.

The example of an embodiment of a first tissue-engagement member 110t illustrated in FIG. 1 is configured to grasp tissue such that the grasped tissue remains in contact with the first tissue-engagement member 110t even if the first tissue-engagement member 110t is moved with respect to the grasped tissue. In some aspects, the first tissue-engagement member 110t illustrated in FIG. 1 is shaped, contoured, configured, etc., to engage tissue in a direction generally transverse to the contact surface of the tissue, and to resist separation from the tissue at least when moved tangentially with respect to the contact surface of the tissue. As used herein, the contact surface of tissue is the outermost layer of the tissue engageable by a component of the tissue-engaging device 100. In the context of a body lumen such as the intestines, the contact surface is the mucosal layer of the tissue wall of the intestines. In some aspects, the first tissue-engagement member 110t illustrated in FIG. 1 is configured to engage tissue in a direction generally transverse to the contact surface of the tissue to grasp the tissue, and to resist separation from the tissue at least when moved in a direction transverse to the direction in which the tissue-grasping feature 112t initially engages with the tissue to grasp the tissue. In some aspects, the first tissue-engagement member 110t includes a tissue-grasping feature 112g shaped, contoured, configured, etc., to remain in contact with grasped tissue even if the tissue-grasping feature 112g is moved with respect to the grasped tissue, such as in a manner described above. In some aspects, the tissue-grasping feature 112g is a tissue-penetrating tip. In some aspects, the tissue-grasping feature 112g has one or more pointed, optionally sharp, terminal ends capable of penetrating tissue (e.g., in a direction transverse to the surface of the tissue). An example of an embodiment of a tissue-grasping feature 112g with a single pointed tip 113 is illustrated in FIG. 1, and an alternative embodiment of a first tissue-engagement member 110t′ with tissue-grasping feature 112g′ with two pointed tips 113′ is illustrated in FIG. 1A. In some aspects, the one or more tissue-grasping features 112g may be curved (e.g., in the form of a hook) to resist movement of the tissue-grasping feature 112g in a direction opposite the insertion direction (e.g., generally transverse to the tissue surface) once inserted into tissue.

In contrast, the second tissue-engagement member 110a illustrated in FIG. 1 is configured simply to engage, but not to also grasp (i.e., hold onto, remain coupled to, etc.), tissue. As such, the second tissue-engagement member 110a does not grasp tissue alone, but only when brought closer to the first tissue-engagement member 110t so that tissue is grasped between the second tissue-engagement member 110a and the first tissue-engagement member 110t. The first tissue-engagement member 110t and the second tissue-engagement member 110a have tissue-contact surfaces 114 which face towards each other to grasp tissue therebetween. In some aspects, the tissue-contact surfaces 114 are generally smooth, atraumatic surfaces which do not penetrate the surface of the grasped tissue. In some aspects, the tissue-contact surfaces 114 may be contoured (e.g., curved, sawtooth patterned, crenulated, or otherwise provided with atraumatic projections which preferably do not penetrate into tissue) to increase the hold on the grasped tissue (e.g., to resist tangential movement between the tissue-contact surfaces 114 and the tissue). In some aspects, the forces exerted by tissue-contact surfaces 114 of the first tissue-engagement member 110t and the second tissue-engagement member 110a against the grasped tissue (generally transverse to the generally planar contact surface of the grasped tissue) are sufficient to hold the grasped tissue in place with respect to the first tissue-engagement member 110t and the second tissue-engagement member 110a such that the grasped tissue is grasped by the tissue-engaging device 100. In such instances, the first tissue-engagement member 110t need not be shaped, contoured, and/or configured to grasp tissue without the aid of another tissue-engagement member.

In some aspects, the second tissue-engagement member 110a has a generally smoothly contoured tissue-contact surface 114 which smoothly transitions to a tissue-engagement feature 112e. The tissue-engagement feature 112e has a sufficiently smooth, rounded, non-sharp, atraumatic, etc., shape, contour, configuration, etc., so that tangential movement of the tissue-engagement feature 112e with respect to tissue (e.g., the contact surface of the tissue) will not injure, damage, or otherwise traumatize the tissue. In other words, an atraumatic tissue-engagement feature 112e does not have features which would penetrate into tissue (e.g., in contrast with the tissue-grasping feature 112t). In some aspects, the tissue-engagement feature 112e has a generally blunt, curved, or otherwise atraumatic distalmost tip.

The above-described features of a tissue-engaging device 100, particularly the tissue-engagement members 110t, 110a, are particularly useful in performing procedures with respect to tissue which require different movements of additional medical devices with respect to target tissue. It will be appreciated that terms such as devices, instruments, tools, etc., may be used interchangeably herein without intent to limit unless otherwise specified. The provision of different components on the same tissue-engaging device 100 allows multiple functions to be performed with the same tissue-engaging device 100 which may otherwise require more than one device. A procedure may thus be performed with fewer devices, thereby simplifying the procedure. It will be appreciated that reference to more than one device includes different devices which are coupled together, such as a traction band coupled with a tissue clip.

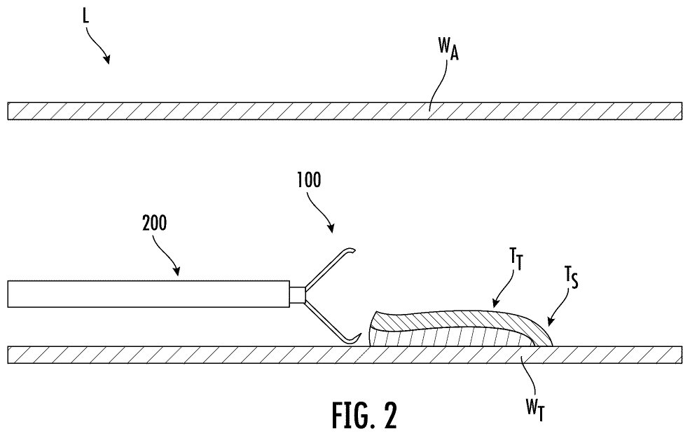

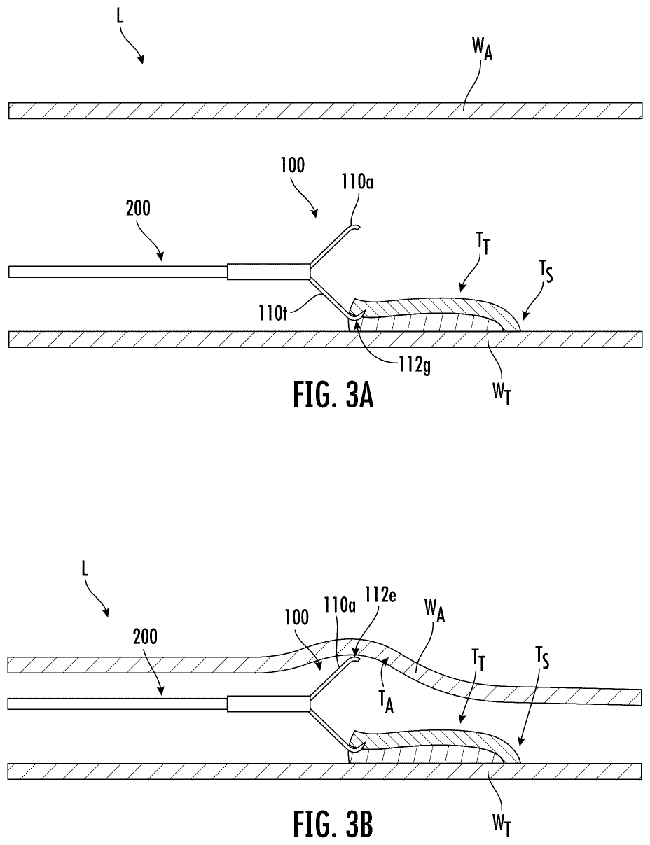

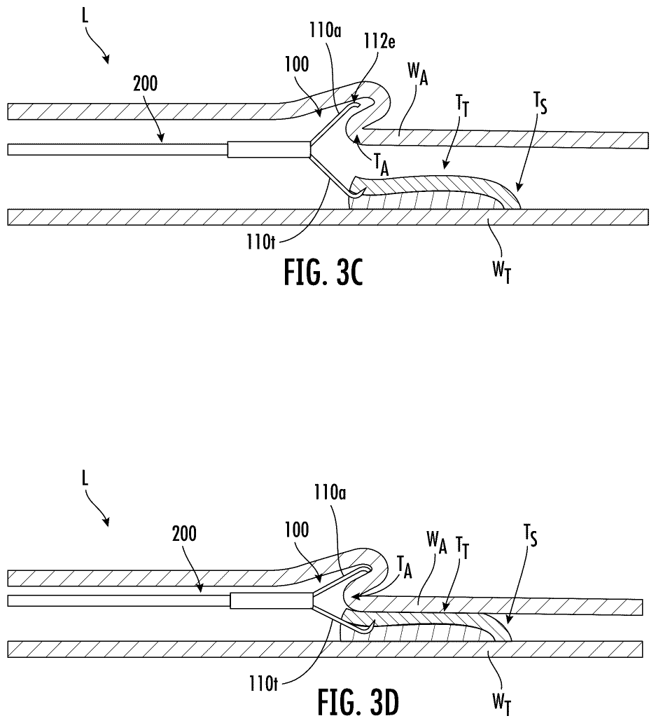

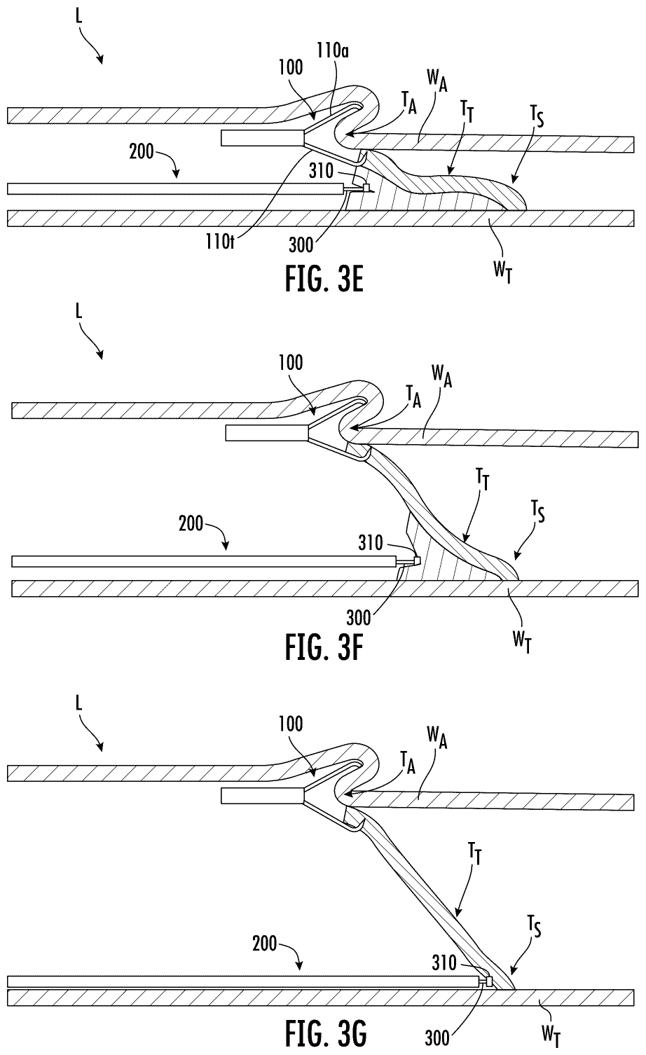

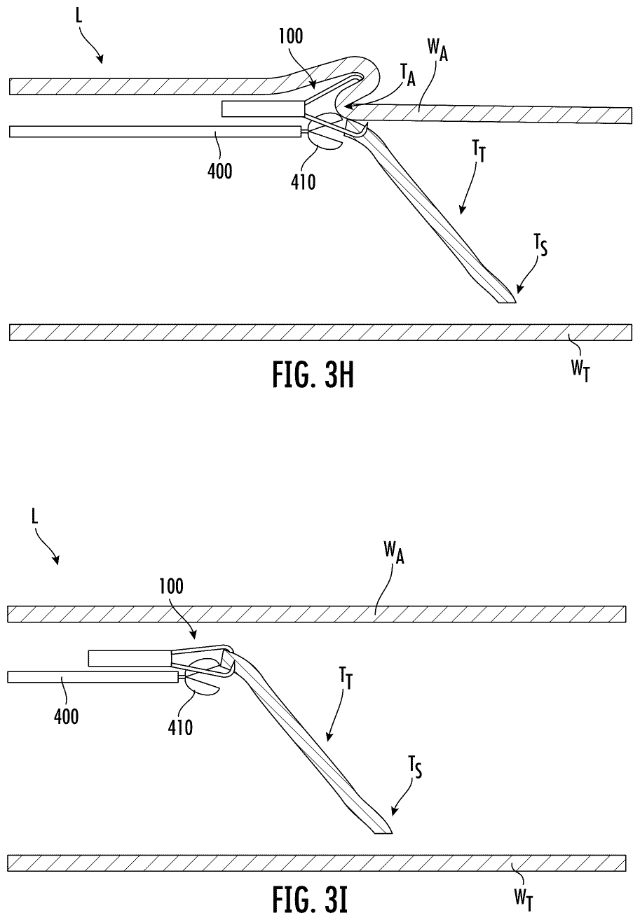

In accordance with various principles of the present disclosure, a tissue-engaging device 100 such as illustrated in FIG. 1 is particularly useful for engaging tissue and applying traction to target tissue being cut, such as dissected by an en bloc ESD procedure as disclosed herein. It will be appreciated that reference is made herein to tissue dissection (and other grammatical forms thereof) for the sake of convenience, such term encompassing tissue dissection, cutting, resection, manipulation, etc. (and other grammatical forms thereof) without intent to limit. An example of a procedure in accordance with various principles of the present disclosure which may utilize and benefit from the features of a tissue-engaging device 100 such as described above is illustrated schematically in FIG. 2 and FIGS. 3A-3I.

As illustrated in FIG. 2, a tissue-engaging device 100 such as illustrated in FIG. 1 may be delivered into a body lumen L with the aid of a delivery device 200. For instance, the tissue-engaging device 100 may be advanced within a lumen defined within the delivery device 200 to be advanced into the body lumen L. In some aspects, the delivery device 200 is an elongate flexible member capable of navigating within a patient's body, such as through tortuous body passages. In some aspects, the delivery device 200 is a simple elongate tubular member such as a catheter. In some aspects, the delivery device 200 is a medical scope (e.g., an endoscope, arthroscope, bronchoscope, colonoscope, cystoscope, duodenoscope, gastroscope, hysteroscope, laparoscope, ureteroscope, etc.), or otherwise includes additional components, such as visualization devices. It will be appreciated that the proportions of the delivery device 200 illustrated in FIG. 2 (and FIGS. 3A-3I) are not necessarily on the same scale as the scale in which the body lumen L and/or tissue-engaging device 100 is illustrated.

The delivery device 200 delivers the tissue-engaging device 100 to be adjacent to (e.g., proximal to) target tissue TT with respect to which a procedure (e.g., resection) is to be performed. As may be appreciated, the target tissue TT is positioned along a target tissue site Ts along a target section WT along a wall of the body lumen L and is surrounded by tissue of the wall of the body lumen L. As may also be appreciated, because the target tissue TT is located within a body lumen L, the tissue wall on which the target tissue TT is located extends circumferentially or annularly so that another section of the wall of the body lumen L is spaced apart from the target tissue TT and may be used as an anchor section WA of the wall of the body lumen L. As may be further appreciated, deflation and insufflation of the body lumen L affect the distance between the target section WT and the anchor section WA of the wall of the body lumen L, and are thus used advantageously in accordance with various principles of the present disclosure as will now be described.

The target tissue may be prepared prior to engagement of the tissue-engaging device 100 therewith (e.g., prior to delivery of the tissue-engaging device 100 thereto) such as to facilitate engagement of a component of the tissue-engaging device 100 therewith. For instance, in Endoscopic Mucosal Resection (EMR) techniques as well as Endoscopic Submucosal Dissection (ESD) techniques, the submucosal layer at/adjacent to the target site is treated with a saline solution, which functions to lift the mucosal layer (in the case of the intestines, the outermost layer of the tissue wall when approached from within, and, thus the contact layer of the tissue wall). The lifting of a layer of the tissue wall forms what is known in the art as a “bleb.” The bleb may facilitate easy access to and visualization of the lesion, and may create a protective barrier at the muscularis layer. In FIG. 2, the body lumen L is illustrated with a bleb below/in the region of the target tissue TT.

The procedure may proceed with the tissue-engaging device 100 being moved into engagement with the target tissue TT, as illustrated in FIG. 3A. It will be appreciated that the body lumen L typically is in an insufflated configuration to facilitate movement and manipulation of the delivery device 200 and the tissue-engaging device 100 within the body lumen L. A first component (e.g., a first tissue-engagement member 110t) of the tissue-engaging device 100 configured to grasp the target tissue TT is advanced into engagement with the target tissue TT to grasp a portion/region of the target tissue TT. Even more particularly, a portion of the first tissue-engagement member 110t of the tissue-engaging device 100, e.g., the tissue-grasping feature 112g, engages a portion/region of the target tissue TT. In some aspects, a cutting device (e.g., knife, scalpel, electrocautery device, forceps, scissors, etc.), is inserted to cut a portion (e.g., a proximal portion) of the target tissue Tr to facilitate grasping of the first tissue-engagement member 110t with the target tissue TT. Because the first tissue-engagement member 110t is configured to grasp tissue, the first tissue-engagement member 110t grasps the target tissue TT to remain therewith (engaged together) as the first tissue-engagement member 110t and/or the target tissue TT moves, such as upon cutting the target tissue TT, as described in further detail below.

The tissue-engaging device 100 typically is in an open configuration, with the first tissue-engagement member 110t and the second tissue-engagement member 110a spaced apart from each other, to facilitate engagement and grasping of the first tissue-engagement member 110t with respect to the target tissue TT, as illustrated in FIG. 3A. The tissue-engaging device 100 may remain in an open configuration to facilitate engagement of a second component of the tissue-engaging device 100 with a portion/location of the body lumen L opposite the target tissue TT, such as illustrated in FIG. 3B. The second tissue-engagement member 110a is extended into contact with a portion/location of the body lumen L selected to function as an anchor section WA during the procedure. The body lumen L may be deflated/desufflated (at least partially) to facilitate extension of the second tissue-engagement member 110a into contact with the anchor section WA while the first tissue-engagement member 110t remains engaged with (and grasping) the target tissue TT, such as illustrated in FIG. 3B.

Deflation of the body lumen L relaxes the anchor section WA such as by reducing tension on the anchor section WA (typically, when the body lumen L is insufflated, the wall of the body lumen L is taut). With the body lumen L deflated (and optionally further deflated from when the second tissue-engagement member 110a of the tissue-engaging device 100 engages the anchor section WA), the wall thereof is not as taut. The second tissue-engagement member 110a of the tissue-engaging device 100 may thus readily (without damaging tissue) fold a section of the anchor section WA, as illustrated in FIG. 3C, such as to define anchor tissue TA. Because the tissue-engagement feature 112e is shaped, contoured, configured, etc., to atraumatically engage tissue, engagement of the tissue-engagement feature 112e with tissue along the anchor section WA of the wall of the body lumen L (even in a direction transverse to the contact surface of the anchor section WA) does not result in puncturing or otherwise penetrating through the wall of the body lumen L. The first tissue-engagement member 110t and the second tissue-engagement member 110a may then be moved closer together, towards a closed configuration, such as illustrated in FIG. 3D. If necessary, the body lumen L is further deflated to allow the tissue-engagement members 110t, 110a to be brought closer together. The anchor tissue TA is thereby grasped between the first tissue-engagement member 110t and the second tissue-engagement member 110a and held with respect to the tissue-engaging device 100. In some aspects, the tissue-engaging device 100 may be released from the delivery device 200 once the tissue-engagement members 110t, 110a are brought close enough to grasp the anchor tissue TA, such as illustrated in FIG. 3E.

With the tissue-engaging device 100 in the closed configuration, the second tissue-engagement member 110a is anchored with respect to the anchor section WA of the wall of the body lumen L with the assistance of the first tissue-engagement member 110t, and the target tissue TT is anchored with respect to the anchor tissue TA. As such, the target tissue TT is anchored with respect to the anchor tissue TA at the anchor section WA at/via/by the tissue-engaging device 100 alone, without an additional element, such as a traction band or other component, between the tissue-engaging device 100 and either the target section WT or the anchor section WA. Such anchoring may be functionally equivalent to prior anchoring techniques using traction bands, but without the need for a traction band or anything other than the tissue-engaging device 100 which engages the target tissue TT to which traction is to be applied during a procedure as well as the anchor tissue TA.

To apply and/or to increase traction to the target tissue TT, the body lumen L may be insufflated to move the anchor section WA away from the target section WT. As illustrated in FIG. 3E, a tissue-cutting device 300 may be advanced to the target tissue TT to dissect, resect, etc., the target tissue TT from the surrounding tissue. In some aspects, tissue adjacent and/or surrounding the target tissue TT (e.g., around the perimeter of the target tissue TT) is cut to separate the target tissue TT and to leave a margin around the target tissue TT which is being removed from the target section WT. As tissue (e.g., submucosal, such as illustrated) is cut at the target section WT, the target tissue TT (e.g., mucosal tissue and optionally also submucosal, as illustrated) separates from surrounding tissue. As may be appreciated, the tension or traction on the target tissue TT is thereby reduced. Further insufflation of the body lumen L as tissue is cut further increases the distance between the anchor section WA and the target section WT, such as illustrated in FIG. 3F. The increased distance between the anchor section WA and the target section WT results in an increase in traction applied to the target tissue TT by the anchor tissue TA and by the tissue-engaging device 100 which remains engaged with (e.g., grasping) both the target tissue TT and the anchor tissue TA. Insufflation of the body lumen L is continued until the full extent (e.g., area, distance, etc., optionally including a margin around the periphery) of the target tissue TT to be removed has been reached by the cutting device 300 and cut, such as illustrated in FIG. 3G.

Once the final cut through the target tissue Tr has been completed, the target tissue TT remains grasped by the tissue-engaging device 100, such as illustrated in FIG. 3H. This is because the tissue-engaging device 100 is configured with a first component (e.g., the first tissue-engagement member 110t and/or the tissue-grasping feature 112g) configured to retain its grasp of tissue even when moved with the anchor section WA as the anchor section WA is drawn away from the target section WT, and even if the first component is moved laterally or tangentially with respect to the target section WT. More particularly, the first component may engage the target tissue TT in a first direction such that movement in a second direction transverse to the first direction does not affect the position and/or grasping of the first component with respect to the target tissue TT and the first component does not move in the second direction. In contrast, the second component of the tissue-engaging device 100 (e.g., the second tissue-engagement member 110a), which is engaged with anchor tissue TA, is configured to releasably engage tissue. More particularly, the second component engages tissue without being restricted from moving with respect to the tissue (e.g., laterally and/or tangentially with respect to the tissue). Restriction of movement of the second component with respect to the tissue it engages is caused by/as a result of another component (e.g., the first component) interacting with the second component (e.g., moving in proximity to the second component to grasp the engaged tissue between the second component and the other component). Therefore, movement of the second component with respect to tissue engaged by the second component (e.g., the anchor tissue TA) results in/causes release of the second component from the engaged tissue without damaging the engaged tissue.

As illustrated in FIG. 3H, a withdrawal tool 400 may be advanced into operable contact with the tissue-engaging device 100. The withdrawal tool 400 may include an end effector 410 configured to grasp the tissue-engaging device 100. Proximal movement of the withdrawal tool 400 to proximally withdraw the tissue-engaging device 100 from the body lumen L moves the first tissue-engagement member 110t (e.g., proximally) with respect to the anchor tissue TA. The shape, contour, configuration, etc., of the first tissue-engagement member 110t and tissue-engagement feature 112e (if provided) allows the tissue-engagement feature 112e to be moved with respect to the anchor tissue TA (e.g., generally tangentially, such as by sliding with respect to the anchor tissue TA) without damaging the anchor tissue TA. The tissue-engaging device 100 is thus released from the anchor tissue TA and the anchor section WA of the wall of the body lumen L without damaging or otherwise affecting the tissue at the anchor section WA of the wall of the body lumen L. The tissue-engaging device 100, along with the target tissue TT, may thus be proximally withdrawn from the body lumen L, such as upon proximal movement of the withdrawal tool 400, such as illustrated in FIG. 3I. As may be appreciated, because the component of the tissue-engaging device 100 which engages the anchor section WA of the wall of the body lumen L engages the anchor section WA atraumatically, the anchor tissue TA may return to its initial configuration with respect to the anchor section WA (prior to being engaged by the tissue-engaging device 100).

As may be appreciated, devices, systems, and methods in accordance with various principles of the present disclosure simplify application of traction to tissue by using a tissue-engagement member which does not require further components, such as traction bands and associated components for coupling the traction band with the tissue-engagement member.

Although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. Moreover, it will be appreciated that although reference is made to use of a tissue-engaging device within a body lumen, other applications are within the scope and content of the present disclosure as may be appreciated by one of ordinary skill in the art. Furthermore, principles of the present disclosure are applicable to other anatomical structures, such as, but not limited, those allowing a tissue-engaging device as formed in accordance with various principles of the present disclosure to extend from a target site to an anchor site. It will further be appreciated that the medical devices used with a tissue-engaging device as described herein may include a variety of medical devices for navigating body lumens, and may be inserted via different access points and approaches, and/or by delivery devices different from the delivery device used to deliver the tissue-engaging device.

It will be appreciated that all apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. It should be apparent to those of ordinary skill in the art that variations can be applied to the disclosed devices, systems, and/or methods, and/or to the sequence of steps of the method described herein without departing from the concept, spirit, and scope of the disclosure. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of tissue-engaging devices and associated systems, as well as various aspects of methods of engaging and/or applying traction to tissue such as described above, and in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A method of applying traction to tissue within a patient's body lumen, said method comprising:
grasping, with the same tissue-engaging device, target tissue at a target section along a wall of the body lumen and anchor tissue at an anchor section along a wall of the body lumen spaced apart from the target section; and
insufflating the body lumen to move the anchor section apart from the target section while the tissue-engaging device remains grasping both sections to apply traction to the target section.

2. The method of claim 1, further comprising cutting tissue adjacent the target tissue to separate the target tissue from surrounding tissue, and further insufflating the body lumen to separate the anchor section further from the target section to increase tension on the target tissue.

3. The method of claim 1, further comprising insufflating the body lumen to advance the tissue-engaging device to the target tissue, and deflating the body lumen sufficiently to allow a first component of the tissue-engaging device to grasp the target tissue and a second component of the tissue-engaging device to engage the anchor tissue.

4. The method of claim 3, further comprising deflating the body lumen sufficiently to allow the second component to form a fold in the anchor section to define the anchor tissue.

5. The method of claim 4, further comprising cutting tissue at the target section to separate the target tissue from surrounding tissue, wherein the first component of the tissue-engaging device is configured to grasp and remain engaged with the target tissue, and the second component of the tissue-engaging device is configured to be slid off the anchor tissue.

6. A method of engaging two different areas of tissue in a patient's body in different manners using the same tissue-engaging device, said method comprising:
grasping a first tissue section with a first component of the tissue-engaging device to grasp the first tissue section in a first direction and to remain engaged therewith when moved transverse to the first direction;
engaging a second tissue section, spaced apart from the first tissue section, with a second component of the tissue-engaging device to engage the second tissue section without restricting movement of the second component with respect to the second tissue section; and
causing movement of the second tissue section to be restricted with respect to the second component with the use of a component of the tissue-engaging device other than the second component.

7. The method of claim 6, further comprising grasping the second tissue section by moving the first component closer to the second component to grasp the second tissue section therebetween.

8. The method of claim 7, further comprising:
cutting the first tissue section away from surrounding tissue; and
removing the tissue-engaging device from the patient's body by sliding the tissue-engaging device with respect to the second tissue section to remove the second component from engagement therewith without damaging the second tissue section, and maintaining the first tissue section grasped by the first component.

9. A tissue-engaging device comprising:
a first component configured to grasp a first tissue section to remain in place with respect to the first component; and
a second component configured to engage a second tissue section while remaining movable tangentially with respect to the engaged second tissue section;
wherein the first component and the second component are movable with respect to each other to move the first component into proximity with the second component to grasp the second tissue section between the second component and the first component to limit tangential movement of the second tissue section with respect to the second component.

10. The tissue-engaging device of claim 9, wherein the first component is shaped to engage tissue in a first direction and to be restricted from moving in a second direction transverse to the first direction.

11. The tissue-engaging device of claim 10, wherein the first direction is transverse to the contact surface of the tissue.

12. The tissue-engaging device of claim 11, wherein the first component is shaped to penetrate the tissue along the first direction.

13. The tissue-engaging device of claim 11, wherein the first component includes a tissue-grasping feature in the form of a tissue-penetrating feature, and the second component has a blunt tissue-engagement feature.

14. The tissue-engaging device of claim 13, wherein the first component is curved.

15. The tissue-engaging device of claim 10, wherein the second component is shaped to remain movable with respect to the second tissue section in both the first direction and the second direction.

16. The tissue-engaging device of claim 9, wherein the first component is defined along a first tissue-engagement member, and the second component is defined along a second tissue-engagement member, the first tissue-engagement member and the second tissue-engagement member being movable with respect to each other between a first configuration in which tissue may be received between the first tissue-engagement member and the second tissue-engagement and a second configuration in which the first tissue-engagement member holds tissue with respect to the second tissue-engagement member to grasp tissue between the first tissue-engagement member and the second tissue-engagement member.

17. The tissue-engaging device of claim 16, wherein the first tissue-engagement member and the second tissue-engagement member are pivotably coupled with respect to each other.

18. The tissue-engaging device of claim 16, wherein the first component is defined along a distal end of the first tissue-engagement member, and the second component is defined along a distal end of the second tissue-engagement member.

19. The tissue-engaging device of claim 16, wherein the first component is shaped to penetrate tissue in a direction transverse to the contact surface of the first tissue section.

20. The tissue-engaging device of claim 19, wherein the first component is shaped to retain the first tissue section grasped therewith upon cutting the first tissue section away from surrounding tissue.
